(19) United States
(12) Patent Application Publication
(10) Pub. No.: US 2023/0132782 A1
(43) Pub. Date: May 4, 2023

(54) METHOD AND COMPOSITIONS FOR TREATING, PREVENTING OR LIMITING THE OCCURRENCE OF VIRAL INFECTION

(71) Applicant: GLOBAL BIOLIFE INC., Bethesda, MD (US)

(72) Inventor: Daryl Lee THOMPSON, Winter Haven (US)

(21) Appl. No.: 17/911,455

(22) PCT Filed: Mar. 16, 2021
(86) PCT No.: PCT/US2021/022538

§ 371 Date: Sep. 14, 2022

Related U.S. Application Data
(60) Provisional application No. 62/990,168, filed on Mar. 16, 2020.

(51) Int. Cl. A61K 31/37; A61P 31/14

(52) U.S. Cl. CPC A61K 31/37; A61P 31/14

(57) ABSTRACT

Method and pharmaceutical compositions for treating or limiting the occurrence of viral infections by administering a therapeutically effective amount of a pharmaceutical composition that targets the ACE2 active site. The pharmaceutical compositions include those of Formula (I), Formula (II), Formula (III), including Formulas (IIIa) and (IIIb), and the viral infections including but not limited to respiratory viruses and disease conditions and syndromes that are associated with the viral infections.

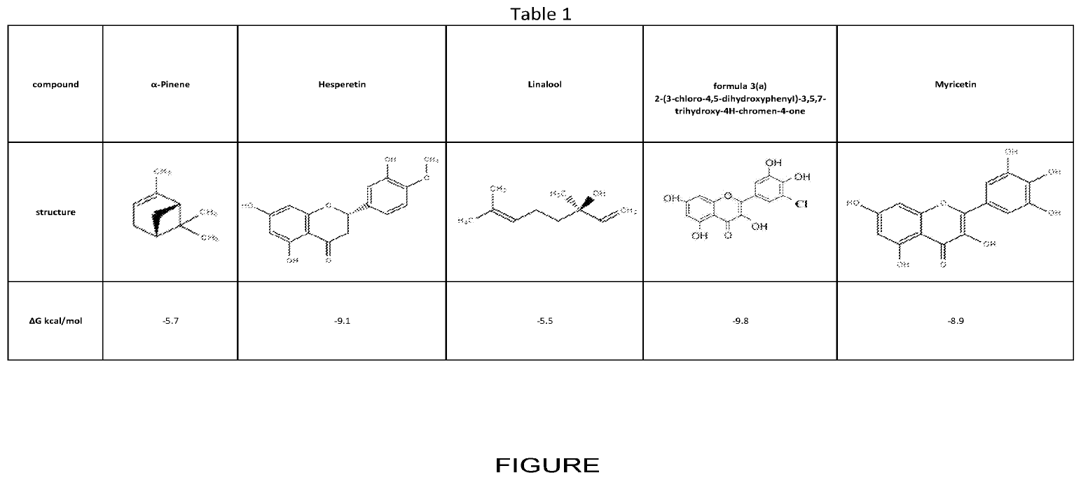

### CROSS REFERENCE TO RELATED APPLICATIONS

This application claims the benefit of priority to U.S. Provisional Application No. 62/990,168, filed Mar. 16, 2020, herein incorporated by reference.

### FIELD OF THE INVENTION

The present invention relates to a method and composition for treating viral infection including a method and composition that targets an Angiotensin Converting Enzyme (ACE2) active site in a host or patient to thereby treat, prevent or limit the occurrence of the viral infection.

### BACKGROUND OF THE INVENTION

Many human diseases result from infection by microscopic organisms called viruses. Infection by viruses can give rise to symptoms that vary from mild to severe. Viral infections can result in large numbers of deaths. Examples of such pandemics include the Spanish flu of 1918-1919 that killed approximately 40 million people and the HIV/AIDS epidemic that has killed almost 2 million people.

Viruses require host organisms in order to replicate and viruses are transmitted from an infected host to an uninfected host through a number of mechanisms. A virus will first attach itself to a host cell. It will then enter the cell and release its genetic code (i.e., RNA or DNA). The virus makes use of the host cell's functional proteins and enzymes in order to replicate. Eventually, the host cell may die because the mechanisms it needs to survive are controlled by the virus. After death of the cell, the replicated viruses are released, allowing them to attack new host cells and continuing the replication process. Some viruses cause modification of the host cells leading to cancer, while other viruses can remain dormant in the host for an extended period prior to the infection becoming symptomatic in the host.

The symptoms that result from viral infections can vary from virus-to-virus as any one virus typically will infect only certain types of cells. This observation also means that a specific virus will typically infect only certain species, although mutation of a virus can allow it to extend the number of species that any one virus is able to infect.

Host species have developed a number of defense mechanisms to protect themselves from viral infections. The first lines of defense are mechanisms that prevent viral entry into the host. The skin provides an impermeable barrier to entry. Viruses typically enter the body through body cavities and can pass through the mucosal surfaces that line these cavities. Once a virus is in the body and detected by the body's immune system, lymphocytes and monocytes in the blood learn how to attack the invader. Invaded cells release cytokines such as the interferons (for example IL 1, IL 6, IL 12, IL 16), tumor necrosis factor (TNF-α), and interferons (typically interferons a and g). The role of these cytokines is to increase the resistance of other host cells to the invading virus. Many of the symptoms of viral infection experienced by the host results from the extensive release of cytokines, commonly referred to as the cytokine storm.

The white blood cells are able to remember how to combat viruses that have previously invaded the body. So if the host survives the initial attack of the virus, the immune system is able to respond much more quickly to subsequent infections of the same virus. The body has developed an immunity to the virus. Such immunity can also be induced by presenting the immune system with a surrogate (vaccine) for the virus in a process known as immunization.

Antiviral drugs are known in the art to assist the immune system in overcoming a viral infection in a patient. Most antiviral drugs work by slowing the replication of the virus in the infected patient's body thus allowing the body's immune system to launch an effective response when the disease symptoms are less severe. Antiviral drugs may work specifically on one or two viruses or may be effective across a broad spectrum of viruses. There are many known mechanisms by which antiviral agents can slow viral replication. One antiviral strategy is to slow or prevent the virus infiltrating a target cell, for example by binding to a receptor on the target cell which is required by the virus to enter the cell or by coating the virus so preventing its ability to bind to the target receptor(s). Other antiviral agents can slow viral replication once the virus particle has entered the target cell. Such mechanisms are well known in the art.

### SUMMARY OF THE INVENTION

The present invention relates to methods and compositions for treating or limiting the occurrence of viral infection using compositions including pharmaceutical compositions that target the Angiotensin Converting Enzyme (ACE2) active site for the treatment, prevention or limiting the occurrence of infection. Advantageously, the infection and disease conditions and syndromes are caused by viruses and the present treatment addresses the disease conditions and syndromes produced by the viral infection. Possible viral infections (and their related disease conditions/syndromes) for which targeting the ACE2 active site treat include but are not limited to respiratory viruses and their related disease conditions or syndromes, such as those caused by coronaviruses including but not limited to COVID-19.

Further, the present invention relates to methods and compositions for treating viral diseases using various formulations which include but are not limited to those of granted U.S. Pat. Nos. 8,034,838; 10,123,991; and 10,383,842 and pending applications U.S. Ser. Nos. 16/302,292 and 16/544,308; all herein incorporated by reference.

The present invention relates to the use of compositions, pharmaceutical agents and the like which are effective antiviral agents to treat, prevent, or limit the occurrence of various infection diseases including viruses such as but not limited to influenza, rhinovirus and coronaviruses.

Although the treatment may include the various compositions and compounds of the aforementioned granted U.S. patents and pending applications (see paragraph [0010] above), therapeutic treatment of individuals suffering from an infection by one or more coronavirus including the COVID-19 coronavirus includes those pharmaceutical agents (compounds and compositions) which the target ACE2 active site. The present method and treatment using the aforementioned compounds and compositions disclosed in the above-cited patents/applications are especially suitable to making an individual less susceptible to a corona infection via ACE2 based on the currently understood way in which the coronavirus is pathogenic.

Referring to the currently perceived pathology of infection of an individual coronavirus, the coronavirus presents a unique challenge in that it appears to exploit a ‘hand shake’ docking site to human cellular membranes that is atypical of influenza and rhinovirus. Influenza attaches to the cell membranes of a host (e.g., human) through the use of ICAM or intercellular adhesion molecules to download (insert) its genetic material. It is now becoming clear that the present strain of coronavirus (i.e., COVID-19) is hijacking the ACE2 or Angiotensin Converting Enzyme pathway to accomplish the same goal. The issue or challenge is that ACE2 is essential for maintaining the health of the pulmonary system and may not be a straightforward target for inhibition.

The compounds and compositions in the aforementioned granted patents and pending applications of paragraph [0010] above including the compounds in the claims of the aforementioned respective patents/applications can be used as therapeutics in the form of molecular probes which make an individual ACE2 less susceptible to the corona infection by modulating.

Compounds of the aforementioned patents and pending applications were shown in advanced computational models to inhibit ACE2 and cause conformational changes of ACE2 on the spike protein, helicase, and protease sites. That renders it less susceptible to interact with coronavirus.

In various advantageous forms, the pharmaceutical composition comprises compounds of Formula I, Formula II, and Formula III including Formula IIIa and Formula IIIb having the chemical structure as follows:

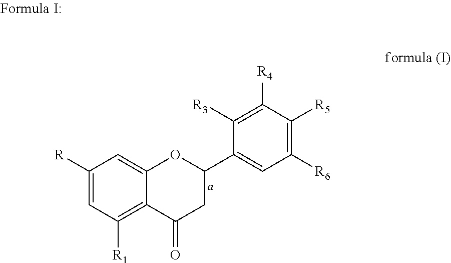

wherein R and R₅ are each independently hydrogen, a hydroxy group, an alkoxy group, a rutinosyl group, and a rhamnosyl group; R₁═OH, R₂═OH, R₃═H, R₄═OH and R₆═OH; and

a is a single bond or a double bond; provided that at least one of R and R₅ comprises an electrophilic group chosen from aldehyde, haloalkane, alkene, butyryl, fluorophenol, sulfonamide and fluorophenyl sulfoxide,

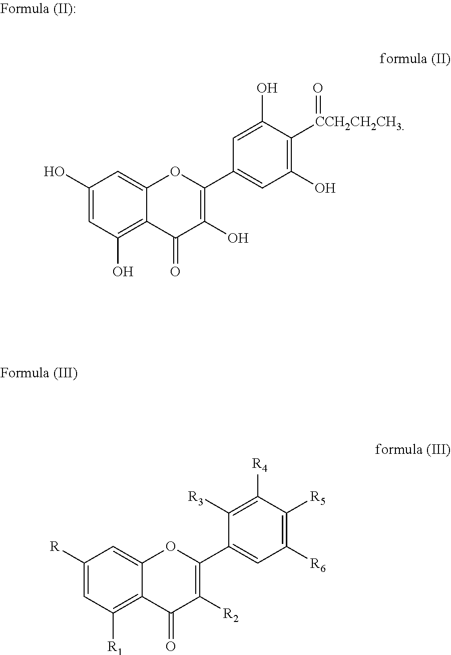

wherein R, R₁, R₂, R₄, R₅, and R₆ are a hydroxyl group or chlorine,

R₃ is hydrogen; and wherein, at least one of R, R₁, R₂, R₄, R₅, and R₆ is chlorine,

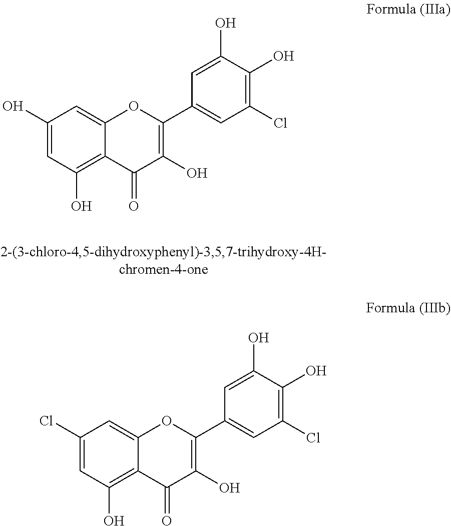 [SMILES: O=c1c(O)c(-c2cc(O)c(O)c(Cl)c2)oc2cc(O)cc(O)c12 ; O=c1c(O)c(-c2cc(O)c(O)c(Cl)c2)oc2cc(Cl)cc(O)c12]

The present invention, in one advantageous form is directed to a method for treating or limiting the occurrence of viral infection and includes administering a therapeutically effective amount of a pharmaceutical composition comprising a compound of Formula I.

The method in one advantageous form includes the composition having an effective amount for treating a virus by targeting the Angiotensin Converting Enzyme (ACE2) active site for the treatment, prevention or limiting occurrence of infection. In one further advantageous form, the viral infection is caused by COVID-19.

The present invention in another advantageous form is directed to a method for treating or limiting the occurrence of viral infection by administering a therapeutically effective amount of a pharmaceutical composition comprising Formula II.

The present invention in yet another advantageous form is directed to a method for treating or limiting the occurrence of a viral infection by administering a therapeutically effective amount of a pharmaceutical composition comprising Formula III.

The present invention in one advantageous form comprises Formula III having a chemical structure selected from Formula IIIa and Formula IIIb.

The present invention in still yet another form is directed to a method of treating viral infection with a therapeutically effective composition selected from the group consisting of Formula I, Formula II, Formula III, Formula IIIa, and Formula IIIb further comprising hesperidin and piperine.

### BRIEF DESCRIPTION OF THE FIGURE

The sole FIGURE is Table 1 for Δ G (kcal/mol) values for binding of select compounds to the ACE2 active site.

### DETAILED DESCRIPTION

The present invention will now be described with reference to specific examples of treatment using the pharmaceutical compositions of the present invention including administering a therapeutically effective amount of the selected compound.

As used herein, the following terms and phrases shall have the meaning set forth below.

The phrase “naturally occurring” when referring to a compound means a compound that is in a form in which it can be found naturally. A compound is not in a form that is naturally occurring if, for example, the compound has been purified and separated from at least some of the other molecules that are found with the compound in nature. A “naturally occurring compound” refers to a compound that can be found in nature, i.e., a compound that has not been created or modified by man.

“Treating” a condition or disease refers to curing as well as ameliorating at least one symptom of the condition or disease.

The term “therapeutic effect” is art-recognized and refers to a local or systemic effect in animals, particularly mammals, and more particularly humans caused by a pharmacologically active substance. The phrase “therapeutically effective amount” means that amount of such a substance that produces some desired local or systemic effect at a reasonable benefit/risk ratio applicable to any treatment. The therapeutically effective amount of such substance will vary depending upon the patient and disease or condition being treated, the weight and age of the patient, the severity of the disease or condition, the manner of administration and the like, which can readily be determined by one or ordinary skill in the art. For example, certain compositions described herein may be administered in a sufficient amount to produce a desired effect at a reasonable benefit/risk ratio applicable to such treatment.

For example, in the context of the therapeutic methods provided in this disclosure, a therapeutically effective amount is an amount that will target the Angiotensin Converting Enzyme (ACE2) active site for the treatment, prevention or limiting occurrence of infection including but not limited to viral infects caused by coronavirus including COVID-19.

The term “pharmaceutically acceptable carrier” means a carrier or diluent that does not give a stimulus to an organism and destroy the natures and bioactivities of an administered compound.

The present method and compositions were identified through research and experimentation to determine compounds which target, i.e., bind or act as an inhibitor bound to human Angiotensin Converting Enzyme-related carboxypeptidas (ACE2). Predictive and empirical A G values for binding compounds to the ACE2 active site demonstrated efficacy of the present compounds to treat and limit viral infections. The Δ G values were measured in terms of kcal/mol noting that the more negative the Δ G value, the higher predictive binding affinity. Table 1 (FIGURE) summarizes Δ G values for select compounds effective for treating viral infections in accordance with the disclosed treatment of viral infections.

The data summarized in Table 1 demonstrates that hesperidin, chlorinated myricetin (compound formula 3(a)), and myricetin are effective in treating viral diseases as targeting the ACE2 binding site. Additional inhibitors of ACE2, i.e., targeting the ACE2 binding site can be identified by conducting similar studies and determining the Δ G value as summarized in Table 1 above and in Table 2 below.

TABLE 2

| | | | Formula | |
|---|---|---|---|---|
| α-Pinene | Hesperetin | Linalool | IIIa* | Myricetin |
| Blind Docking | | | | |
| −6.2 | −6.2 | −5.9 | −7.9 | −7.3 |
| −5.9 | −5.9 | −5.8 | −7.9 | −7.3 |
| −5.7 | −5.7 | −5.7 | −7.8 | −7.3 |
| −5.7 | −5.7 | −5.6 | −7.7 | −7.3 |
| −5.7 | −5.7 | −5.5 | −7.7 | −7.2 |
| −5.6 | −5.6 | −5.4 | −7.7 | −7.2 |
| −5.6 | −5.6 | −5.3 | −7.7 | −7.2 |
| −5.5 | −5.5 | −5.2 | −7.5 | −7.2 |
| −5.5 | −5.5 | −5.2 | −7.5 | −7.2 |
| MERS Pocket Docking | | | | |
| −5.9 | −6.8 | −5.3 | −6.6 | −6.5 |
| −5.6 | −6.4 | −5.2 | −6.3 | −6.2 |
| −5.6 | −5.6 | −5.1 | −5.9 | −6.1 |
| −5.5 | −5.3 | −5.1 | −5.8 | −5.7 |

It will now be clear to a person of ordinary skill in the art that the present compounds are effective for treating various viral infections by acting as ACE2 inhibitors binding to the ACE2 active site. Additional compounds relating to the disclosed compounds a pharmaceutical compositions disclosed here can be used and easily adapted for treating viral infections by identifying those compounds which have affinity for the ACE2 active site.

Although the invention has been described above in relation to preferred embodiments thereof, it will be understood by those skilled in the art that variations and modifications can be accomplished in these preferred embodiments without departing from the scope and spirit of the invention.

1. A method for treating or limit the occurrence of viral infection, said method comprising administering a therapeutically effective amount of a pharmaceutical composition comprising formula (I) 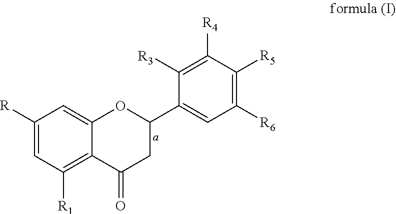
wherein R and R₅ are each independently hydrogen, a hydroxy group, an alkoxy group, a rutinosyl group, and a rhamnosyl group; R₁═OH, R₂═OH, R₃═H, R₄═OH and R₆═OH; and
a is a single bond or a double bond; provided that at least one of R and R₅ comprises an electrophilic group chosen from aldehyde, haloalkane, alkene, butyryl, fluorophenol, sulfonamide and fluorophenyl sulfoxide,
wherein said viral infection is caused by COVID-19.

2. The method of claim 1, wherein the composition is effective for the treatment of a virus by targeting the Angiotensin Converting Enzyme (ACE2) active site for the treatment, prevention or limiting occurrence of infection.

3. The method of claim 2, wherein the viral invention is caused by COVID-19.

4. A method for treating or limit the occurrence of viral infection, said method comprising administering a therapeutically effective amount of a pharmaceutical composition comprising formula II

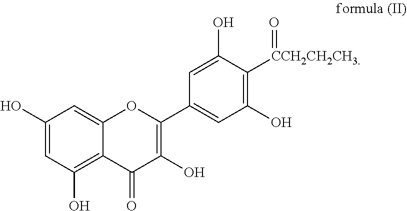

5. The method of claim 4, wherein the composition is effective for the treatment of a virus by targeting the Angiotensin Converting Enzyme (ACE2) active site for the treatment, prevention or limiting occurrence of infection.

6. The method of claim 5, wherein the viral invention is caused by COVID-19.

7. A method for treating or limit the occurrence of viral infection, said method comprising administering a therapeutically effective amount of a pharmaceutical composition comprising formula (III) 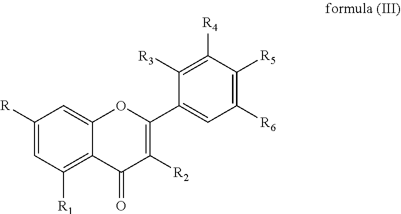
wherein R, R₁, R₂, R₄, R₅, and R₆ are a hydroxyl group or chlorine,
R₃ is hydrogen; and wherein, at least one of R, R₁, R₂, R₄, R₅, and R₆ is chlorine, and
wherein said viral infection is caused by COVID-19.

8. The method of claim 7, wherein the composition is effective for the treatment of a virus by targeting the Angiotensin Converting Enzyme (ACE2) active site for the treatment, prevention or limiting occurrence of infection.

9. The method of claim 8, wherein the viral invention is caused by COVID-19.

10. The method of claim 7, wherein formula 3 is selected from the compounds:

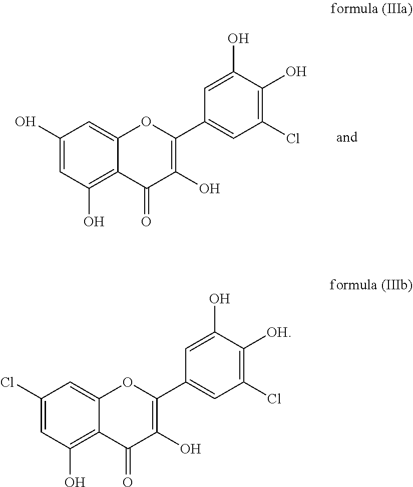 [SMILES: O=c1c(O)c(-c2cc(O)c(O)c(Cl)c2)oc2cc(O)cc(O)c12 ; O=c1c(O)c(-c2cc(O)c(O)c(Cl)c2)oc2cc(Cl)cc(O)c12]

11. The method of claim 1, wherein the pharmaceutical composition further comprises hesperidin and piperine.

12. A method of limiting the occurrence of, reducing the risk or severity of or treating viral infections comprising administering a composition consisting of therapeutically effective amounts of a pharmaceutical composition comprising myricetin and hesperitin to a patient at risk of or diagnosed with viral infection wherein the composition targets the Angiotensin Converting Enzyme (ACE2) active site for the treatment, prevention or limiting occurrence of the viral infection.

13. The method of claim 12, wherein the viral infection is caused by a coronavirus.

14. The method of claim 13, wherein the coronavirus is COVID-19.

15. The method of claim 12, wherein about 300 to about 700 mg myricetin and about 100 to about 500 mg hesperitin are present in the composition.

16. The method of claim 12, wherein about 450 to about 600 mg myricetin and about 250 to about 400 mg hesperitin are present in the composition.

17. The method of claim 12, wherein about 55 to about 75% weight myricetin and about 30 to about 50% hesperitin based on the total weight of the mixture, is present in the composition.

18. The method of claim 12, wherein the ratio of myricetin to hesperitin present in the composition is about (30-60):(30-60).
